(19) United States
(12) Patent Application Publication
(10) Pub. No.: US 2008/0160115 A1
(43) Pub. Date: Jul. 3, 2008

(54) Method, substance and device to prevent the flow of mother's milk

(76) Inventors: Keren Shaked, Cholon (IL); Uzi Ezra Havosha, Tel Aviv (IL)

Correspondence Address: UZI EZRA HAVOSHA & PARTNERS, HADAR-DAFNA HOUSE 39 SHAOUL HAMELECH STREET, TEL AVIV, 64928, omitted

(21) Appl. No.: 11/645,898

(22) Filed: Dec. 28, 2006

(51) Int. Cl. A61K 36/31; A41C 3/12

(52) U.S. Cl. 424/755; 450/57

(57) ABSTRACT

Extracts, in the form of cream, ointment and the like, from cabbage leaves to reduce or to prevent the flow of mother's milk when applied to the mother's breast. A pad with such extracts to fit on a breast. Vacuum sealed pads.

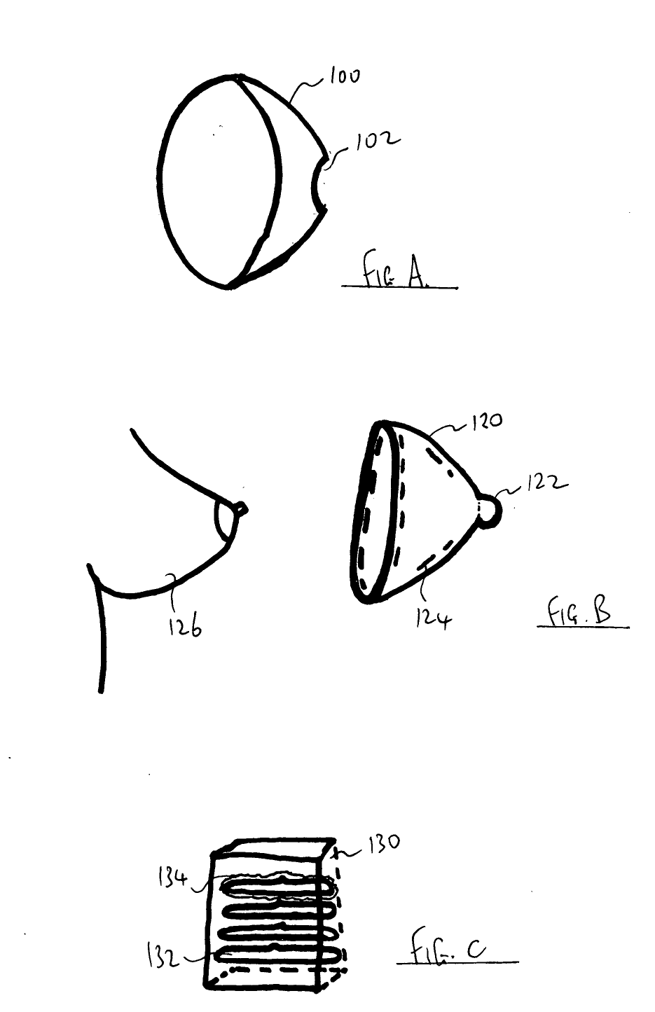

### FIELD OF THE INVENTION

This invention is in the field of mother's milk and in particular the control of the flow of the milk.

### BACKGROUND OF THE INVENTION

From the birth of a child for approximately two years, the mother's breasts produce milk that can be used to feed the child.

There can be many reasons why a mother may wish to reduce and even stop the flow of milk. A common reason is when a mother produces too much milk in the first few days after giving birth and the baby is not yet ready to drink such quantities. This causes a build up of excess milk in the breast and a discomfort for the mother called engorgement. It is well known in the art that the leaves of the cabbage plant placed on the mother's breast, relieve this discomfort after a few hours. There are various methods of application recommended. Some say to cool the cabbage leaves before applying and some say also to pierce or crush the leaves before applying. The application can be to hold a leaf on each breast for between half an hour up to two hours or to place the leaves under the brassier or bra.

All opinions seem to agree that the system works and warn that if done “too much”, say for more than three or four times a day for more than three or four days then the milk supply will begin to reduce. This warning is given to mothers who want to reduce the pain of engorgement but do not want to reduce noticeably the amount of milk production. Ice packs and other treatments are known to have a similar effect but the cabbage treatment is considered to be the most effective.

Prolonged use of the cabbage treatment will cause the milk production to wane and eventually to dry up. This is the second application of this invention. Mothers may wish to dry up their milk supply for a variety of reasons, for example, after a stillbirth, the body hormones still cause milk to be produced, and the mother does not have a baby to feed. Also a mother who has been breast feeding and wishes to go to work or is separated from her child for any other reason, will continue to produce milk and not have an outlet for it.

Breast pumps are effective to take out excess or unused milk but the more that is pumped the more milk is produced. It does not stop the milk flow. It has the opposite effect. Such pumps are used by mothers wanting to increase their milk production or wishing to continue breast feeding after an interruption. Emptying the breasts of their milk is what keeps milk production going. A baby's sucking or a pump pumping sends messages to the brain, which then releases the hormone oxytocin. Oxytocin causes the muscle cells around the alveoli to contract, pushing milk down through the ducts to the nipple. This movement of milk down the ducts is called the milk-ejection reflex.

When the alveoli are empty, they respond by making more milk. Recent research suggests that a special protein in human milk, called feedback inhibitor of lactation (FIL), regulates milk production (Wilde 1995). When there is a lot of milk in the breast, FIL inhibits, or prevents, the alveoli from making more. When milk is removed from the breast—and FIL is not there to stop milk production—the alveoli get busy and manufacture more milk.

The above explains why breast pumps are not the solution for reducing the milk supply. One of the best known solutions is the cabbage treatment. Unfortunately the traditional cabbage treatment is cumbersome, awkward, time consuming, odorous, and can be embarrassing. For example, a lady who is in the company of others in a formal environment like at work, may have to disappear frequently to change the cabbage leaves under her bra.

This invention comes to relieve this unfortunate situation.

Whereas the method of using cabbage for this purpose is well known, the reasons for it working are still largely unknown. The cabbage leaf contains a number of ingredients including sinigrin, magnesium, oxylate, mustard oil, rapine and sulphur heterosides. It is thought that this 20 combination of compounds reduces tissue congestion by dilating the small blood vessels to improve blood flow to the area. This seems to send the message to the alveoli to stop producing milk.

Even though the effect of the cabbage is generally known, no-one has utilized this knowledge to make a substance that answers the needs of ladies in the twenty-first century who, for whatever the reason, wish to reduce or stop their milk production. There is a need for a convenient method of application of such a substance. This invention answers this need.

### SUMMARY OF THE INVENTION

It is to be understood that both the foregoing general description and the following detailed description present embodiments of the invention, and are intended to provide an overview, or framework, for understanding the nature and character of the invention as it is claimed. The accompanying drawing is included to provide a further understanding of the invention, and is incorporated into and constitute a part of this specification. The drawing illustrates various embodiments of the invention and, together with the description serve to explain the principles and operations of the invention.

The object of this invention is to make a substance that can controllably reduce the amount of milk produced by a lactating mother.

Another object of this invention is to make a substance that can stop the milk produced by a lactating mother.

Another object of this invention is to make a substance to reduce or stop the production of milk and that can be conveniently carried and applied.

This invention involves processing the cabbage plant in a number of ways to produce a cream, a gel, a liquid or any other form that enables the convenient application of the substance so made.

Care is taken not to affect or damage the natural ingredients of the cabbage. Only those parts of the cabbage are removed that prevent the convenient application of the substance. The essential ingredients remain and are effective in carrying out their job of reducing and/or stopping mother's milk.

The process of extracting the cabbage includes at least one of the following; boiling, freezing, crushing, grinding and freeze drying.

In another embodiment, cabbage that has been treated as above or any combination of the said processes and/or with the addition of other substances or processes, with the aim that the end product is a cream, ointment, gel or liquid. An object of this invention is to make a suitable pad one side impervious and other side with the prepared substance and optionally with cut-out for nipple protection, or alternatively a plastic sheet layer with the ability to exude the substances in the pad to the other side of the plastic sheet layer namely the woman's breast.

Another object of this invention is for the pad to have layers of material including polymer substances or other absorbing materials to hold the cabbage extracts before being exuded onto the woman's breast.

An object of this invention is to make a pad to put inside a bra optionally with a sticky substance on the outer surface of the pad to hold the pad in place in the bra and so that the cream etc and/or other substances would be exposed to the skin of the breast. The said pad could be made with the sticky substance on part of the pad facing the skin to independently stick on a milk-producing breast thereby enabling the pad and its creams etc. to be in contact with the breast without having to use a bra to hold it in place. This pad would be impregnated with the above mentioned cream etc and/or other substances in order to impart the said cream etc and/or other substances directly to the skin of the breast.

Another object of this invention is to have the said pad in vacuum packaging before being opened for use.

Another object of this invention is to have a cut-out section in the said pad at the pointed end to expose the nipple of the woman's breast and to prevent undue pressure on the said nipple with the option of having a removable cap optionally made of a firm convex material to cover the nipple and optionally to have liquid absorbing material in that cover or in that part of the pad where no such cut-out section or cover is used.

Another object of this invention is to make cream or other substances that are made with natural ingredients.

Another object of this invention is to include vitamins and/or minerals to the said creams and/or other substances.

### BRIEF DESCRIPTION OF THE DRAWINGS

The accompanying drawing, which is incorporated in and forms a part of this specification, illustrate embodiments of the invention and, together with the description, serves to explain, by way of example only, the principles of the invention:

FIG. A is a drawing of one embodiment of the pad of this invention.

FIG. B is a drawing of another embodiment of the pad of this invention.

FIG. C is a drawing of an example of packaging for the said pads.

### DESCRIPTION OF THE PREFERRED EMBODIMENTS

As will be appreciated the present invention is capable of other and different embodiments than those discussed above and described in more detail below, and its several details are capable of modifications in various aspects, all without departing from the spirit of the invention.

Accordingly, the drawing and description of the embodiments set forth below is to be regarded as illustrative in nature and not restrictive.

FIG. A shows a breast pad 100 of this invention. It is shaped to fit the contours of the woman's breast. It could be impregnated with any combination of substances mentioned above and with, optionally, a vacuum packaging to preserve the freshness of the active ingredients prior to being opened and used.

There is a circular cut-out 102 to expose the nipple of the breast.

FIG. B shows the pad 120 with a nipple protector 122. This nipple protector 122 could be an optional additional feature that would protect the nipple from damage caused by rubbing or pressure when clothes are worn over the pad. When the baby starts to feed from the said nipple, the nipple protector 122 optionally could be lifted to expose the nipple while making it unnecessary to remove the pad. The nipple protector 122 could be made of a firm substance that would help to protect the nipple.

The pad 120 could have sticky substance 124 on its inside surface to help it stick on the woman's breast 126. In the absence of this sticky substance 124 the pad 120 would probably be kept in place by the woman's bra. There could be a sticky substance on the outside surface of the pad to keep the pad in place in the bra.

FIG. C shows a possible packaging 130 of the pad 132 of this invention where many pads 132 stacked one on top of the other. The pads may be vacuum packed 134 to preserve the freshness of the substances in the pad 132.

The present invention is not intended to be limited to the embodiments described above, but to encompass any and all embodiments within the scope of the following claims.

What is claimed:

1. A pad made to fit the shape of a woman's breast made of materials that absorb substances in such a way that they enable the application of the said substances to surfaces desired to be in contact thereto where the said substances include leaves of the cabbage plant and extracts therefrom whereby a lactating mother's milk production is reduced.

2. A pad made to fit the shape of a woman's breast partly made of materials that absorb substances in such a way that they enable the application of the said substances to surfaces desired to be in contact thereto where the said substances include leaves of the cabbage plant and extracts therefrom and partly made of materials that absorb other substances whereby a lactating mother's milk production is reduced by means of contact between the said cabbage leaves and extracts therefrom and the said breast and whereby mother's milk that comes out from the said breast is absorbed into the said pad.

3. A pad as claimed in claims 1 and 2 wherein the said substances include one or more of the following substances namely crushed cabbage leaves, extracts from the said leaves, alcoholic based compounds used inter-alia for the purpose of preservation of the freshness of the said substances and compounds to impart a desired smell.

4. A pad as claimed in claims 1 and 2 where the production of mother's milk is stopped.

5. A pad as claimed in claims 1 and 2 wherein the said substances include the extracts resulting from one or more of the following processes namely heating, boiling, freezing, crushing, grinding, freeze drying, and distilling the said cabbage leaves.

6. A pad as claimed in claims 1 and 2 wherein the said substances are in the form of one or more of the following formats namely an ointment, a cream, a gel, a liquid and pieces of cabbage leaves.

7. A pad as claimed in claims 1 and 2 wherein the said substances include vitamins.

8. A pad as claimed in claims 1 and 2 wherein the said substances contain only natural ingredients.

9. A pad as claimed in claims 1 and 2 further comprising other substances combined with the said cabbage leaves for preservation of freshness, ease of application for example, alcohol based compounds, creams, gels and/or aromatic substances.

10. A pad as claimed in claims 1 and 2 that is stored in a vacuum package with at least one pad in the said package.

11. A pad as claimed in claims 1 and 2 that is multi-layered with layers of material that absorb substances and layers of material that exude substances.

12. A pad as claimed in claims 1 and 2 made into a frustum shape open at its base and top by removing the apex of the cone thereby exposing the nipple of the breast.

13. A pad as claimed in claim 12 with a removable and replaceable cap over the said open top for protection of the nipple of the said breast from contact with, for example, clothing and to protect the nipple area from contact with the said cabbage leaves or the said extracts therefrom.

14. A pad as claimed in claim 12 further comprising absorbent material in the apex of the said pad for absorbing any liquid that drips from the said breast such absorbent material being of the kind that prevents the absorbed liquid from remaining in contact with the surface of the said nipple or breast.

15. A pad as claimed in claims 1 and 2 that is a suitable size and shape to fit inside a woman's bra.

16. A pad as claimed in claim 15 that has a sticky substance on its outer surface to better hold it in place in the said bra.

17. A pad as claimed in claims 1 and 2 that is made to attach to a woman's breast, with the aid of sticky substances on the side of the said pad that touches the said breast.

18. A substance made from cabbage leaves that has the effect to reduce the flow of milk from a lactating mother's breast when the said substance is applied to the said breast.

19. A substance as claimed in claim 18 that includes one or more of the following substances namely cabbage leaves, extracts from the said leaves, alcoholic based compounds used inter alia for the purpose of preservation of the freshness of the said substances and compounds to impart a desired smell.

20. A substance as claimed in claim 19 where the said extraction is made by including one or more of the following processes namely heating, boiling, freezing, crushing, cutting, grinding, freeze drying, and distilling the said cabbage leaves crushing grinding boiling or freezing.
